SEQUENCE LISTING (14 sequences)

SEQ ID NO: 1 - protein, 9 aa, Human Cytomegalovirus
NLVPMVATV

SEQ ID NO: 2 - protein, 13 aa, Artificial Sequence
  PADRE epitope
AKXVAAWTLKAAA

SEQ ID NO: 3 - protein, 13 aa, Tetanus
YIKANSKFIGITE

SEQ ID NO: 4 - protein, 14 aa, Tetanus
VSTIVPYIGPALNI

SEQ ID NO: 5 - protein, 25 aa, Artificial Sequence
  K25V peptide
KSSAKXVAAWTLKAAANLVPMVATV

SEQ ID NO: 6 - protein, 28 aa, Artificial Sequence
  KTet830V fusion peptide
KSSYIKANSKFIGITEAAANLVPMVATV

SEQ ID NO: 7 - protein, 26 aa, Artificial Sequence
  KTet639V fusion peptide
VSTIVPYIGPALNIAAANLVPMVATV

SEQ ID NO: 8 - DNA, 20 bp, Artificial Sequence
  DNA adjuvant
tccatgacgttcctgacgtt

SEQ ID NO: 9 - DNA, 23 bp, Artificial Sequence
  DNA adjuvant
tcgtcgttttgtcgttttgtcgt

SEQ ID NO: 10 - DNA, 19 bp, Artificial Sequence
  DNA adjuvant
gggggacgatcgtcggggg

SEQ ID NO: 11 - DNA, 41 bp, Artificial Sequence
  PCR primer
cagtcagctagcgtttaaacatgcagatcttcgtgaagacc

SEQ ID NO: 12 - DNA, 47 bp, Artificial Sequence
  PCR primer
ggacaacggcgaccgcgcgactccctaccccccctcaagcgcaggac

SEQ ID NO: 13 - DNA, 47 bp, Artificial Sequence
  PCR primer
gtcctgcgcttgagggggggtagggagtcgcgcggtcgccgttgtcc

SEQ ID NO: 14 - DNA, 34 bp, Artificial Sequence
  PCR primer
ccgggtacctcaacctcggtgctttttgggcgtc

(19) United States
(12) Patent Application Publication
(10) Pub. No.: US 2004/0101534 A1
(43) Pub. Date: May 27, 2004

(54) Adjuvant-free peptide vaccine

(76) Inventor: Don J. Diamond, Glendora, CA (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/603,094

(22) Filed: Jun. 25, 2003

Related U.S. Application Data
(60) Provisional application No. 60/391,088, filed on Jun. 25, 2002.

(51) Int. Cl. A61K 39/12; A61K 39/245; A61K 39/25

(52) U.S. Cl. 424/186.1; 424/230.1

(57) ABSTRACT

Linking the synthetically-derived T helper epitope, PADRE, or one of several tetanus T_{H} epitopes to the immunodominant HLA A*0201-restricted CTL epitope from CMV-pp65 in a fusion peptide caused robust cytotoxic cellular immune responses in HLA A*0201/K^{b} transgenic mice. The fusion peptides are immunogenic when administered in saline solution by either subcutaneous or intranasal routes. CpG-containing single-stranded DNA (ss-ODN), when added to the fusion peptides as an adjuvant, dramatically upregulated immune recognition by either route. Target cells which either expressed full length pp65 protein from vaccinia viruses or were sensitized with the CTL epitope encoded in the vaccine were recognized by splenic effectors from immunized animals. T_{H}-CTL epitope fusion peptides in combination with CpG ss-ODN (DNA adjuvant) represents a strategy useful for parenteral or mucosal delivery of vaccines in a safe and effective manner that has applicability for control or prophylaxis of infectious disease, especially in situations such as vaccination of donors or recipients of HCT, where highly inflammatory adjuvants are not desired.

This application claims the benefit of U.S. Provisional Application No. 60/391,088, filed Jun. 25, 2002.

### GOVERNMENT RIGHTS STATEMENT

This invention was made with government support from the United States Public Health Service under Grant Nos. CA 77544, CA 30206-Project 3, A1 44313, and A1 43267, SAIC Subcontract #20×S192A, and a Core grant to the City of Hope Cancer Center (CA 33572). The National Institutes of Health, National Cancer Institute (DTP) also supported this research. The United States government may have certain rights in the invention.

### BACKGROUND OF THE INVENTION

1. Technical Field

This invention generally relates to the field of immunology and in particular to vaccines. Adjuvant-free peptide vaccines, according to an embodiment of this invention, modify immune reaction to human cytomegalovirus. Vaccines may be administered with an adjuvant, which preferably is a DNA adjuvant.

2. Description of the Background Art

Investigators have focused on developing transgenic mice containing human leukocyte antigen (HLA) alleles such as A*0201 (A2.1) in the Class I system, or DR1 in the Class II system to address the problem of selection of epitopes that bind to major histocompatibility complex (MHC) molecules in an experimental model system. Bernhard et al., J. Exp. Med. 168:1157-1162, 1998; Rosloniec et al., J. Exp. Med. 185:1113-1122, 1997. In these model systems, workers can study cellular immune responses to vaccines in an easily manipulated vertebrate system with immunologic similarities to humans. A repertoire of CTL epitopes specific for the immunodominant protein, CMV-pp65 from human cytomegalovirus (CMV), have been characterized. Wills et al., J. Virol. 70:7569-7579, 1996; Longmate et al., Immunogenetics 52:165-173, 2001. Although CMV is a significant opportunistic infection in solid organ transplant recipients, hematopoietic cell transplant (HCT) recipients and HIV patients and causes numerous congenital problems in the fetus and infant, there remains no Federal Food and Drug Administration (FDA)-approved vaccine against CMV. HLA-restricted CTL epitopes are useful as vaccines because HCT recipients are HLA-typed and therefore can be selected for potential response to an HLA-restricted epitope-based vaccine. Furthermore, reactivation of CMV and viremia are monitored routinely during the first several months after HCT. HCT recipients therefore represent a convenient opportunity to investigate the properties of a therapeutic vaccine. Nichols et al., Blood 97:867-874, 2001; Zaia et al., Hematology 339-355, 2000; Krause et al., Bone Marrow Transplant 19:1111-1116, 1997.

Since CMV-infected cells express pp65 both early and late in infection, pp65 is an appropriate vaccine target. Grefte et al., J. Gen. Virol. 73:2923-2932, 1992; Riddell et al., J. Immunol. 146:2795-2804, 1991. Vaccines incorporating pp65 peptides provide a method to immunize against CMV infection in the clinical setting. Since CMV-pp65 contains an HLA A2.1-specific epitope that is recognized by T cells from both HLA A2.1 humans and mice of the H-2^{b} background containing an HLA A2.1 or chimeric (human/mouse) A2.1/K^{b} transgene, this peptide epitope, pp65₄₉₅₋₅₀₃ (SEQ ID NO:1) is a model Class I epitope for these studies. Wills et al., J. Virol. 70:7569-7579, 1996; Diamond et al., Blood 90:1751-1767, 1997; BenMohamed et al., Immunology 106:113-121, 2002. To circumvent a need for allele specificity for the required T_{H} epitope, a series of T_{H} sequences that promiscuously bind to either human or murine Class II MHC alleles have been evaluated in combination with the CMV-pp65 HLA A*0201-restricted epitope. Diamond et al., Blood 90:1751-1767, 1997; BenMohamed et al., Hum. Immunol. 61:764-779, 2000.

In the last decade, investigators have studied many methods to deliver peptides corresponding to either CTL or T_{H} epitopes in experimental vaccines. For example, peptides have been emulsified in adjuvants, complexed to alum or suspended in liposomes. Hioe et al., Vaccine 14:412-418, 1996; Mora et al., J. Immunol. 161:3616-3623, 1998; Partidos et al., J. Immunol. Meth. 206:143-151, 1997. Successful epitope vaccine strategies against virus, bacterial, and tumor antigens have been developed in mice using these delivery vehicles. Hart et al., Proc. Natl. Acad. Sci. U.S.A. 88:9448-9452, 1991; Shirai et al., J. Immunol. 152:549-556, 1994. However, most of these strategies are not suitable for use in humans.

Modification of the primary structure of peptides with lipids has been extensively studied both in experimental animals and man. Livingston et al., J. Immunol. 159: 1383-1392, 1997; Martinon et al., J. Immunol. 149:3416-3422, 1992. Lipopeptides (lipidated peptides) specific for heptatitis B (HBV), HIV, and tumor antigens have been studied clinically in phase 1 and 2 trials, but with only modest results. Heathcote et al., Hepatology 30:531-536, 1999; Gahery-Segard et al., J. Virol. 74:1694-1703, 2000; Seth et al., AIDS Res. Hum. Retrovirus 16:337-343, 2000. Exposure of ex-vivo expanded dendritic cells to peptides also has proven to stimulate cellular immunity more effectively than many parenteral vaccination regimes. Banchereau et al., Cancer Res. 61:6451-6458, 2001; Ludwig et al., J. Virol. 72:3812-3818, 1998. However, a stable small molecule product for vaccination is much simpler and therefore preferable to methods requiring cell isolation.

Adjuvants, especially those which are oil-based or contain mycobacterial components may be used in animals, yet in many cases are too inflammatory for human use. Most vaccine protocols use adjuvants which localize the antigen to a physical site (‘depot effect’) and provoke generalized immune response pathways. Oil based, pro-inflammatory adjuvants such as Freunds' Complete Adjuvant (FCA) can cause ulceration in immunized animals. While a number of adjuvant compositions are known in the field (e.g. aluminium hydroxide, liposomes or squalene) they each have features or biochemical properties (e.g irritants) that limit their broad applicability. Indeed, only aluminium hydroxide has been approved by the FDA for use in humans. Therefore, the methods to deliver vaccines without adjuvants that are nevertheless effective would be highly desirable. Freytag et al., Curr. Top. Microbiol. Immunol. 236: 215-236, 1999; Newman et al., Vaccine 15:1001-1007, 1997; Wiedmann et al., J. Pathol. 164:265-271, 1991; Belyakov et al., Nat. Med. 7:1320-1326, 2001.

Ideally, a vaccination protocol should be able to safely elicit a strong, persisting immune response. Furthermore, vaccine administration involving sensitive tissues or mucosa (e.g. ocular or intra-nasal delivery) may preclude use of adjuvants that elicit tissue inflammation (“inflammatory adjuvants”). An effective CMV vaccine employing processed T cell epitopes currently is not available. Plasmid DNA vaccines are for the most part ineffective and live viruses have serious safety concerns. Krieg et al., Proc. Natl. Acad. Sci. U.S.A. 95:12631-12636, 1998; Boyer et al., J. Infect. Dis. 181:476-483, 2000; Berger et al., J. Virol. 75:799-808, 2001.

Alternative means to enhance effectiveness of subunit protein and peptide vaccines using DNA CpG SS-oligodeoxynucleotide (ODN) adjuvants have been reported in mice and primates. However, several studies have demonstrated that ss-ODN, especially with CpG motifs, skew the immune response to a T_{H}1-dominated one. Davis et al., J. Immunol. 160:870-876, 1998; Homer et al., J. Immunol. 167:1584-1591, 2001. Therefore a need exists in the art for vaccines that are simple to produce and administer, that are effective without an adjuvant and particularly without a classical inflammatory adjuvant, yet produce a robust cytotoxic response.

### SUMMARY OF THE INVENTION

Accordingly, this invention relates to peptide fusions that are useful as vaccines. These fusions comprise a T helper (T_{H}) epitope fused to a CMV CTL epitope and may be administered by different routes, for example mucosally or subcutaneously, either alone or preferably with a DNA adjuvant.

An embodiment of this invention provides a cytomegalovirus vaccine which comprises a fusion peptide composed of a T helper epitope fused to a CMV CTL epitope peptide. Further embodiments provide a fusion peptide comprising a T helper epitope fused to a CMV CTL epitope peptide and a method of modifying the immune response of a mammal to CMV comprising administering an effective amount of a vaccine as discussed above.

### BRIEF DESCRIPTION OF THE DRAWINGS

FIG. 1 provides cytotoxicity data for splenocytes immunized with the doses of K25V peptide, (KSSAKXVAAWTLKAAANLVPMVATV; SEQ ID NO:5), shown.

FIG. 2 provides cytotoxicity (2A) and interferon-gamma (IFN-γ) production (2B) data for immunized splenocytes after one or two in vitro stimulations (IVS).

FIG. 3 provides cytotoxicity data for splenocytes immunized with the doses of K25V peptide with CpG-containing ss-ODN (DNA adjuvant).

FIG. 4 shows comparative cytotoxicity data for splenocytes immunized with KTet₈₃₀V fusion peptide (KSSYIKANSKFIGITEAAANLVPMVATV; SEQ ID NO:6) with and without ss-ODN (DNA adjuvant).

FIG. 5 shows comparative cytotoxicity data for splenocytes immunized with KTet639V fusion peptide (VSTIVPYIGPALNIAAANLVPMVATV; SEQ ID NO:7) with and without ss-ODN (DNA adjuvant).

FIG. 6 provides cytotoxicity data for splenocytes immunized with K25V fusion peptide along, with non-CpG-containing ss-ODN or CpG-containing ss-ODN.

FIG. 7 provides flow cytometry results for splenocytes immunized as described in FIG. 6.

FIG. 8 shows cytotoxicity data for splenocytes immunized once with the indicated immunogen.

FIG. 9 shows cytotoxicity data for splenocytes immunized twice with the indicated immunogen.

FIG. 10 shows cytotoxicity data for a bulk spleen cell culture derived from the K25V immunization described for FIG. 1 after repeated (5×) in vitro stimulation with K25V. The target cells were infected with vaccinia virus expressing the fusion peptides shown.

FIG. 11 shows cytotoxicity data for splenocytes immunized with 50 nmol Tet₆₃₉V alone or with 25 μg CpG ss-ODN against targets expressing the indicated antigens.

### DETAILED DESCRIPTION OF THE PREFERRED EMBODIMENTS

Embodiments of this invention involve the direct modification CMV antigens to enhance immunogenicity which can be effective even without an adjuvant. The addition of a DNA adjuvant to the modified peptides further enhances immunization and also supports alternative dosing routes such as intranasal. Covalently linking selected CTL and T_{H} epitopes creates effective immunogens, even without lipid modification. These highly soluble unlipidated fusion peptides can be administered by any conventional route, for example parenterally or intranasally, in a solution of normal saline and small amounts of dimethyl sulfoxide (DMSO). Fusion of the peptides results in enhanced immunogenicity; the component CTL and T_{H} epitopes are inactive alone when administered without adjuvant.

Published accounts suggest that subcutaneous administration of peptides without adjuvant or lipidation induces sub-optimal immunity, except in rare instances. Sauzet et al., Vaccine 13:1339-1345, 1995: Schild et al., Eur. J. Immunol. 21:2649-2654, 1991. Adjuvant is needed to obtain immune responses in the model used here when PADRE or Tet₈₃₀₋₈₄₃ T_{H} and the pp65₄₉₅₋₅₀₃ CTL epitope peptides are administered as separate peptides in normal (0.9%) saline (data not shown). See BenMohamed et al., Hum. Immunol. 61:754-779, 2000.

To increase immunogenicity, this invention provides vaccines in which both epitopes are fused to create a single peptide. The initial fusion peptide sequence to be evaluated contained the T_{H} epitope PADRE and the pp65₄₉₅₋₅₀₃ CTL epitope, referred to as K25V in Table I. Standard algorithms suggest that the significant hydrophobicity of K25V may enhance membrane association and entry into cellular protein degradation pathways. See Tsunoda et al., Vaccine 17675-685, 1999.

Each of three alternative T_{H} epitopes together with an immunodominant CTL epitope from CMV-pp65 (HLA A2.1) were fused to produce an effective fusion peptide vaccine, the activity of which was further augmented by CpG ss-ODN adjuvant. Any T helper epitope known in the art may be used with the present invention, for example T helper epitopes derived from hepatitis B virus, human immunodeficiency virus-1, CMV pp65, or other epitopes derived from the heavy chain of tetanus toxoid, however the three exemplary epitopes shown in Table I are preferred. Other advantageous T helper epitopes include the following peptides from tetanus heavy chain: 590-603, 615-629, 639-652, 830-843 and 947-967. CMV CTL epitopes are known in the art. Any of these may be used with this invention, however the following peptide epitopes are preferred: A*1101(pp65₁₃₋₂₄); B*0702 (pp65₄₁₇₋₄₂₆ or pp65₂₆₅₋₂₇₅); A*0101 (pp65₃₆₃₋₃₇₃); A*2402 (pp65₃₆₉₋₃₇₉); B*3502 (pp65₁₈₈₋₁₉₅); pp65₁₈₆₋₁₉₆; and pp65₃₆₇₋₃₇₉. The CMV CTL epitope NLVPMVATV (pp65₄₉₅₋₅₀₃; SEQ ID NO:1) is most preferred. Transgenic mice expressing HLA A*1101/K^{b} recognized fusion peptides combining an HLA A*1101-restricted epitope from CMV-pp65 and either the T_{H} epitope, PADRE, or a tetanus derived T_{H} epitope.

The responses to the fusion peptides were augmented by CpG ss-ODN, resulting in a powerful systemic immune response when administered intranasally. Without wishing to be bound by theory, it is believed that mucosal admininstration facilitates processing of the peptide. CpG ss-ODN produces a synergistic response with several different types of fusion peptides. Table I provides the sequences of three exemplary fusion peptides. See Alexander et al., Immunity 1:751-761, 1994; Livingston et al., J. Immunol. 159:1383-1392, 1997; Reece et al., J. Immunol. 151:6175-6184, 1993; and Longmate et al., Immunogenetics 52:165-173, 2001 for discussions of the fusion peptide moieties PADRE, Tet₈₃₀₋₈₄₃, Tet₆₃₉₋₆₅₂ and the CMV CTL epitope, respectively. Non-CpG form DNA adjuvant had minimal additional CTL-stimulating ability beyond the peptide alone in these assays. HLA tetramer reagents that bind to pp65₄₉₅₋₅₀₃ epitope-specific CD8 lymphocytes can be used to determine the numbers of CTL that are stimulated after immunization with fusion peptides and DNA adjuvant. The cytotoxic activity measured by chromium release assay can be correlated with the absolute frequency of CD8 lymphocytes detected by the epitope-specific HLA tetramer reagent. This type of immune response analysis can be used to evaluate the capacity of a peptide vaccine to stimulate the immune system in clinical applications such as HCT or solid organ transplantation.

TABLE I

Primary Structure of CMV Vaccine Peptides.
(X=cyclohexylalanine).

| | | | T_{H} Epitope | | CMV CTL | |
|---|---|---|---|---|---|---|
| | | | Sequence | | Epitope | |
| | Adaptor | | (SEQ ID | | (SEQ ID | HLA |
| | Sequence | T_{H} Type | NOS:2,3,4) | Linker | (NO:1) | Restrict |
| K25V | KSS | PADRE | AKXVAAWTLKAAA | none | MLVPMVATV | A*0201 |
| (SEQ ID) | | | | | | |
| NO:5) | | | | | | |
| KTet₈₃₀V | KSS | Tetanus | YIKANSKFIGITE | AAA | NLVPMVATV | A*0201 |
| (SEQ ID | | | | | | |
| NO:6) | | | | | | |
| Tet₆₃₉V | none | Tetanus | VSTIVPYIGPALNI | AAA | NLVPMVATV | A*0201 |
| (SEQ ID | | | | | | |
| NO:7) | | | | | | |

A therapeutic CMV vaccine for HCT recipients functions to modify CMV immunity during the reconstitution phase (the time-frame of immuno-incompetence) to combat the increased risk for developing CMV disease. In the context of this invention, modifying the immune response to CMV denotes changing the intensity of the cellular and/or humoral response (and preferably both) to one or more CMV epitope. Therefore, an effective amount of a vaccine is an amount that modifies the immune response to the antigen in question. The term immunogenic, therefore, refers to a substance that is able to modify the immune response to that substance. In the context of a vaccine, the durability of CTL memory is important. In immunocompetent transgenic mice, 50% of the original response level to a dilipidated vaccine comprising K25V (Table I) was detected 6 months later (data not shown). Recent evidence suggests that CMV-antigenemia drives the frequency of CMV-specific CTL (as monitored by HLA tetramers). Prolongation of T-help responses is associated with maintenance of CMV-specific CTL. Gratama et al., Blood 98:1358-1364, 2001; Cwynarski et al, Blood 97:1232-1240, 2001; Walter et al., N. Engl. J. Med. 333:1038-1044, 1995; Einsele et al., Blood 99:3916-3922, 2002. The transgenic mice do not have a source of antigen to maintain the response, in contrast to patients who are infected with CMV, therefore, even greater longevity of responsiveness likely will occur after peptide immunization of humans. CD4 responses to fusion peptides of this invention are substantial (S.I.>10) when using the fusion peptide as the recall antigen. Joined T_{H} and CTL epitopes are potent antigens. Whether non-cognate CD4 T_{H} also are an advantage in maintaining CMV-specific CTL has not been determined previously.

pp65 was modified to enhance degradation because unmodified full length pp65 was not efficiently recognized by epitope-specific murine CTL. Apparently the transporter associated with antigen processing TAP-positive antigen presenting cells do not generate sufficient CTL epitope since the T2 TAP-negative target is well-recognized when processed minimal peptide (e.g. pp65₄₉₅₋₅₀₃) is provided. This might be the result of inefficient processing of the unmodified full-length protein, or the 10-fold lower cell-surface HLA A2.1 found on transgenic mouse cells compared to endogenous MHC Class I molecules.

Ubiquitination of pp65 coupled with substituting an N-terminal arginine residue (Ub-R-modification) reduces the T½ of the protein to less than 20 minutes, a change in T½ compared to unmodified pp65 of more than fifty fold. This may explain the greater ability of targets which are infected with Ub-R-pp65Vac to present sufficient cognate CTL epitope to be recognized by murine CTL after fusion peptide immunization. Further, human CTL clones of 5 different haplotypes that recognize pp65 lysed targets more efficiently when they are infected with Ub-R-pp65Vac compared to unmodified pp65Vac.

This report shows that subcutaneous or mucosal (e.g., intranasal) immunization is feasible in a clinical setting. Individuals such as HCT donors can easily tolerate immunization with these preparations, since both peptides and CpG DNA have limited toxicity, especially compared to other oil or mycobacterial-based adjuvants, to amplify the CMV-pp65-specific memory CTL response pre-transplant. Infusion of T-cell replete bone marrow from an immunized donor with the usual “contamination” with mature T cells provides CMV-specific T cells (adoptive immunotherapy). The longevity, however, of donor T cells transferred with either stem cells or bone marrow in the recipient to protect against CMV disease has not been determined, especially in the context of steroid treatment of graft versus host disease. Substituting vaccination for ganciclovir prophylaxis and/or therapy could improve survival after HCT or organ transplant, because the adverse effects of anti-viral chemotherapy would be eliminated. The incidence of late CMV disease might also be decreased, since delayed immune reconstitution caused by the immunosuppressive properties of ganciclovir would be eliminated.

CpG ss-ODN further augments the activity of fusion peptides, providing a safe means to lower the amount given during an immunization while maintaining effectiveness. Healthy adults, children, recipients of either solid organ or hematopoietic transplants or other at-risk individuals may be vaccinated with fusion peptides because there are limited side-effects expected using the formulation. Doses of vaccine peptide suitable for first vaccination are about 50 μg to about 100 mg and preferably about 1 mg to about 25 mg. Boosters may be given if desired at the same dose or lower, and generally are given at intervals of about two to about eight weeks or preferably about four weeks. Anywhere from one to four booster immunizations may be given.

The vaccines may be formulated in any manner known in the pharmaceutical arts, including with no adjuvant, but preferably contain a DNA adjuvant. Preferred DNA adjuvants contain CpG motifs as described in Kreig et al., Curr. Opin. Mol. Ther. 3:15-24, 2001 and Krieg, Annu. Rev. Immunol. 20:709-760, 2002, the disclosures of which are hereby incorporated by reference. Other DNA adjuvants may be used as well, for example bacterial DNA, and other organismic DNAs which do not contain methylated CpG motifs. Most preferred are forms of synthetic DNA which have the phosphorothioate substitution, although the phosphodiester linkage also is possible, but in many situations is less stable to degradation. Preferred DNA adjuvants include 5′-TCCATGACGTTCCTGACGTT-3′ (SEQ ID NO:8; CpG ODN 1826), 5′-TCGTCGTTTTGTCGTTTTGTCGT-3′ (SEQ ID NO:9; CpG ODN 2006), CpG ODN 7909, 5′-GGGGGACGATCGTCGGGGG-3′ (SEQ ID NO:10; CpG ODN 2216), any synthetic DNA sequence which contains two or more CpG motifs separed by 1-10 nucleotides and is repeated at least twice in a 18-25 nucleotide sequence that preferably contains a phosphorothioate linkage, or minimally a phosphodiester linkage. Any CpG DNA sequence which interacts with Toll-like receptor 9 as an agonist is a preferred sequence.

Vaccine formulations preferably include pharmaceutically acceptable carriers suitable for the route of administration being used. Examples of carriers which may be used include saline, saline with small amounts of DMSO (for example 30% or less), water, compatible oils or phosphate-buffered saline, heat shock proteins and proteins or lipid moieties that promote a depot effect of the antigen to allow it to be taken up by antigen presenting cells or dendritic cells. Such formulations are well known in the art and can be modified according to the route of administration, which may include mucosally (for example intranasally, buccally, rectally, vaginally, sublingually, etc.), transdermally, subcutaneously, intradermally, intraperitoneally, intramuscularly, or any known method.

An embodiment of this invention provides refinements of peptide structure and delivery mechanisms to deliver a rational approach for therapeutic vaccines against CMV infection, for example in the context of HCT. A donor that is a suitable match and is clinically acceptable for a candidate for hematopoietic stem cell transplantation will be provided three injections of the fusion peptide vaccine either with or without CpG DNA at 5, 3 and 1 week prior to the start of infusion of stem cells to the recipient. The donor of the transplant often is given granulocyte-colony stimulating factor to increase the quantity of stem cells; however we have not found this treatment to affect their T cell repertoire. A recipient of 70 kg body weight can expect to receive between 0.57-5.7×10⁸ CMV-specific T cells as part of the stem cell infusion. Recipients generally receive one injection post-transplant at day +28 as a booster. Recipients are followed for CMV blood infection and disease, as well as other indicators of procedure-related morbidity and CMV-specific cellular immune responses. Individuals who are not treated with ganciclovir are considered vaccine successes because they do not develop sufficient CMV viremia to require ganciclovir therapy.

The results from in vitro stimulation of human PBL using the pp65₄₉₅₋₅₀₃ peptides shown in Table I confirm that pp65₄₉₅₋₅₀₃ stimulates a CTL precursor (CTLp) memory response in cells from individuals with prior CMV exposure. See Diamond et al., Blood 90:1751-1767, 1997; La Rosa et al., Blood 97:1776-1786, 2001; Villacres et al., J. Infect. Dis. 184:256-267, 2001. A transgenic HLA A2.1 mouse model was evaluated to test whether the peptide could also stimulate de novo CTLP without prior virus exposure. Transgenic mice that had been co-immunized with the PADRE T_{H} epitope in IFA exhibited a robust CTL response directed at the pp65₄₉₅₋₅₀₃ CTL epitope. Diamond et al., Blood 90:1751-1767, 1997; Alexander et al., Immunity 1:751-761, 1994.

These results were confirmed using a mouse (C57BL/6) expressing an HLA transgene modified by substitution of the human α3 domain with the murine homologue (A2/K^{b}) used in these studies. See BenMohamed et al., Hum. Immunol. 61:764-779, 2000. pp65₄₉₅₋₅₀₃-specific CTL stimulation was dependent on T_{H} peptide co-immunization in combination with an adjuvant such as incomplete Freund's adjuvant, although several different T_{H} epitopes including those from tetanus or PADRE work equally well. BenMohamed et al., Hum Immunol. 61:764-779, 2000.

It remained to be shown whether antigen processing in transgenic HLA A2.1/K^{b} mice also would allow recognition of the pp65₄₉₅₋₅₀₃ epitope in the context of a full length protein. Transgenic mice were infected with a vaccinia virus expressing recombinant CMV pp65 (pp65Vac) that had been previously shown to cause recognition of human antigen presenting cells by CMV-specific T cell clones. Diamond et al., Blood 90:1751-1767, 1997. Splenocytes from the infected mice did recognize human T2 target cells pulsed with the CTL epitope, pp65₄₉₅₋₅₀₃ (data not shown). See Longmate et al., Immunogenetics 52:165-173, 2001. Thus, a pp65-specific CTL epitope is specifically recognized in transgenic mice by endogenous processing of full length pp65 protein. This transgenic mouse model is well-recognized in the art as proving results that predict results in humans in the clinical setting.

### EXAMPLES

### Example 1

### Immunogen Construction

pp65₄₉₅₋₅₀₃ (SEQ ID NO:1), the PADRE and tetanus (Tet) T_{H} epitopes (BenMohamed et al., Hum. Immunol. 61:764-779, 2000 and Alexander et al., Immunity 1:751-761, 1994, the disclosures of both of which are hereby incorporated by reference) were prepared by standard solid phase F-Moc procedures using an Applied Biosystem 432 (Foster City, Calif., USA) instrument. Peptides were purified by standard HPLC methods (>90%), and the molecular weight of the peptides was confirmed by matrix-assisted laser desorption/ionization (MALDI) (Kratos, Chestnut Ridge, N.Y.), according to known methods. See La Rosa et al., Blood 97:1776-1786, 2001. Fusion peptides were made available under the auspices of the Rapid Access to Intervention Development (RAID) program (DTP, NCI), including K25V, PAM-K25V, diPAM-K25V, and KTet₈₃₀V (Table I), at purities ≧90%. Tet₆₃₉V (SEQ ID NO:7) was synthesized by Mixture Sciences (La Jolla, Calif.). Incomplete Freund's adjuvant was purchased from Sigma (St. Louis, Mo.).

The previously described (Lipford et al., Eur. J. 1 mm unol. 27:2340-2344, 1997; Krieg et al., Nature 374:546-549, 1995) immunostimulatory synthetic oligodeoxynucleotide (ODN) 1826 (5′ TCCATGACGTTCCTGACGTT 3′; SEQ ID NO:8) containing two CpG motifs (underlined) was synthesized with a nuclease-resistant phosphorothioate backbone by Alpha DNA (Montreal, Quebec, Canada). Sodium salts of the ODN was resuspended at 5 mg/ml in 10 mM Tris (pH 7.0)/1 mM EDTA and stored as 50 μl aliquots at −20° C. The DNA adjuvant was diluted in normal saline prior to injection.

### Example 2

### Recombinant Vaccinia Virus Constructs

The human ubiquitin (Ub) gene (Tobery and Siliciano, J. Exp. Med. 185:909-920, 1997) was amplified using the following pair of primers: 5′ primer A: CAGTCAGCTAGCGTTTAAACATGCAGATCTTCGTGAAGACC (SEQ ID NO:11) and 3′ primer B: GGACAACGGCGACCGCGCGACTCCCTACCCCCCCTCAAGCGCAGGAC (SEQ ID NO:12). HCMV (AD169) pp65 gene was amplified using the following pair of primers: 5′ primer C: GTCCTGCGCTTGAGGGGGGGTAGGGAGTCGCGCGGTCGCCGTTGTCC (SEQ ID NO:13) and 3′ primer D: CCGGGTACCTCAACCTCGGTGCTTTTTGGGCGTC (SEQ ID NO:14). Primers B and C were designed to not only complement each other, but also contain the arginine codon (AGG) to replace methionine (ATG) at the amino terminus of pp65. The Ub gene (271 bp) and HCMV pp65 gene PCR products (1680 bp) were fused together to generate the Ub-(R)-pp65 fusion gene by PCR using the primer pair A and D. The PCR reaction conditions were one cycle at 94° C., 5 min; 5 cycles of 94° C., 1 min, 55° C., 1 min, 72° C., 4 min, followed by 20 cycles of 94° C., 1 min, 60° C., 1 min and 72° C. for 4 min. The resulting 1926 bp Ub-R-pp65 fusion gene product was gel purified and cloned into pSC11 insertion plasmid using Nhe I and Kpn I site to generate Ub-R-pp65-pSC11. Chakrabarti et al., Mol. Cell. Biol. 5:3403-3409, 1985. The construct was verified by restriction enzyme digestion and DNA sequencing. The Ub-R-pp65 recombinant vaccinia virus (Ub-R-pp65Vac) was generated by transfecting the Ub-R-pp65-pSC11 plasmid into VV infected Hu TK⁻ cells. Ub-R-pp65Vac was simultaneously screened and selected for three rounds by color reaction of substrates (Bluogal™, Sigma-Aldrich) to β-galactosidase and resistance to BrdU according to known methods. Diamond et al., Blood 90:1751-1767, 1997. Expression of pp65 was detected by western blot as previously described. See Yao et al., Vaccine 19:1628-1635, 2001.

### Example 3

### Dose Responsiveness of K25V Fusion Peptide

HLA-A2.1/K^{b} transgenic mice used throughout this study were bred and maintained under standard pathogen-free conditions. The expression of HLA-A2.1/K^{b} molecules was routinely confirmed by flow cytometric analyses of splenocytes from individual mice, using BB7.2 monoclonal antibody. See BenMohamed et al., Hum. Immunol. 61:764-779, 2000.

Groups of six- to 9-wk old transgenic mice were immunized with synthetic peptides with or without ss-ODN or with vaccinia viruses. Vaccinia virus (107 pfu) or synthetic peptides were injected using a 1 ml tuberculin syringe (Becton Dickinson & Co., Franklin Lakes, N.J., USA) in a volume of 100 μl of normal saline solution with DMSO without anesthesia at the base of the tail for the subcutaneous route. For intranasal administration, mice received anesthesia with 30 mg/kg intraperitoneal ketamine/xylazine cocktail (Sigma, St. Louis, Mo.) prior to treatment. A total of 301 μl (15 μl/nares) of synthetic peptides with or without ss-ODN in saline solution were administered using a pipette. For some cases, transgenic mice were boosted two weeks later with the same synthetic peptide/DNA combination.

Twelve days after immunization, spleens were aseptically removed and splenic single cell suspensions were produced by teasing the organs through a sterile nylon mesh according to known methods. Splenocytes were stimulated in vitro once or twice with syngeneic antigen presenting cells, loaded with the relevant CMV-CTL epitope. The methods of Diamond et al. (Blood 90:1751-1767, 1997) were modified as follows. Stimulator cells for in vitro stimulations were syngeneic naive splenocytes pre-treated for 3 days with 25 pg/ml lipopolysaccharide (Sigma) and 7 μg/ml dextran Sulfate (Sigma), at a density of 2×10⁶ cells/ml. See Vitiello et al., Eur. J. Immunol. 27:671-678, 1997. The lipopolysaccharide blasts (25×10⁶ cells/100 pl were stimulated) with 100 μM of CMV CTL epitope fusion peptide for 4 hours in a 37° C. 5% CO₂ incubator. Spleens were pooled from each group of immunized mice and the splenic suspensions (3×10⁶ cells) were co-cultured for 7-8 days with 10⁶ γ-irradiated (2400 rad, Isomedix Model 19 Gammator, Nuclear Canada, Parsippany, N.J.) peptide loaded blasts in 2 ml medium containing 10% T-Stim™ Culture Supplement (Collaborative Biomedical Products, Bedford, Mass., USA).

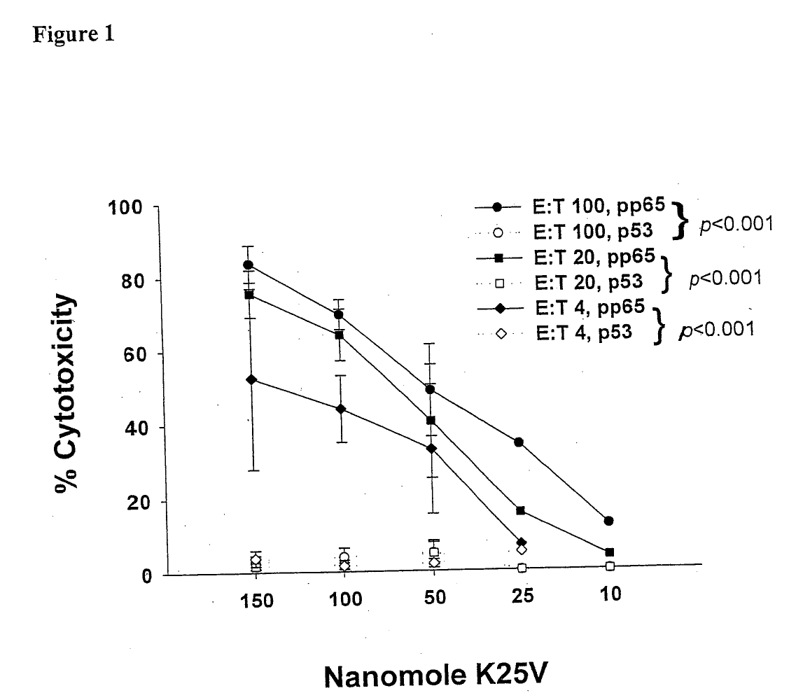

Dose-response was studied by administering K25V subcutaneously in 99% N-saline/1.0% DMSO(NSD) to transgenic mice at several different concentrations of peptide. The results are shown in FIG. 1. K25V was dissolved at 5 mM in 90% N-saline/10% DMSO, and diluted in N-saline to deliver the amount of peptide shown on the X-axis of FIG. 1. Transgenic HLA A2.1/K^{b} (N=6 (150 nmol), 14 (100 nmol), 8 (50 nmol), and 2 (10 and 25 nmol)) mice were immunized once subcutaneously at the base of the tail with peptide and no additional adjuvant. After two weeks, spleens were harvested, and the splenocytes were stimulated in vitro as described above. Targets were T2 cells loaded with specific (pp65₄₉₅₋₅₀₃, filled symbols) and non-specific (P⁵³149-1571 open symbols) peptides. Means and standard error were calculated at each effector:target ratio (E:T) for all evaluated mice, and significant p-values are indicated. CTL activity decreased in a dose-dependent manner between 10 and 150 nanomoles (p<0.001 compared to control peptide). In contrast, immunization with mixtures of T_{H} and CTL epitopes were inactive when injected under the same conditions as the fusion peptide (data not shown).

### Example 4

### Chromium Release Assay

The cytotoxic activity of the cell cultures was determined by a standard 4 hour chromium release assay following one or two in vitro stimulations. To measure peptide-specific responses in HLA A2.1/K^{b} mice, T2 cells (the TAP deficient human cell line, see Wei and Cresswell, Nature 356:443-446, 1992) were pulsed with 10 μM of the relevant peptide or an equal concentration of an unrelated, control synthetic sequence for 1 hour. Recognition of virally-encoded CMV pp65 was evaluated using either Jurkat HLA A2.1 transfectants (Diamond et al., Blood 90:1751-1767, 1997) or HLA A2.1 (La Rosa et al., Blood 97:1776-1786, 2001) EBV-LCL infected overnight at MOI 3 with vaccinia virus according to published protocols. Target cells were labeled with 200 μCi of Na⁵¹CrO₄ (ICN, Costa Mesa, Calif.) for 1 hour in a 37° C. water bath, washed extensively and plated in 96-well round-bottom plates at a concentration of 2000 target cells/well. The radioactivity in the supernatants was determined using a Cobra II™ auto γ-counter (Packard, Downers Grove, Ill., USA), and percent specific lysis was determined as described in La Rosa et al., Blood 97:1776-1786, 2001. Determinations were performed in triplicate, and assay data were taken in consideration only if spontaneous release was <30%. Results were reported when the average and standard deviation of experimental determinations were <15% of the mean. Comparisons of CTL activity using specific versus non-specific peptides or of different conditions within an assay were done using the Student's T test using SigmaPlot™ and SigmaStat™ software (SPSS, Chicago, Ill.). P-Values ≦0.05 were considered significant.

### Example 5

### γ-IFN Detection after Fusion Peptide Inmunization

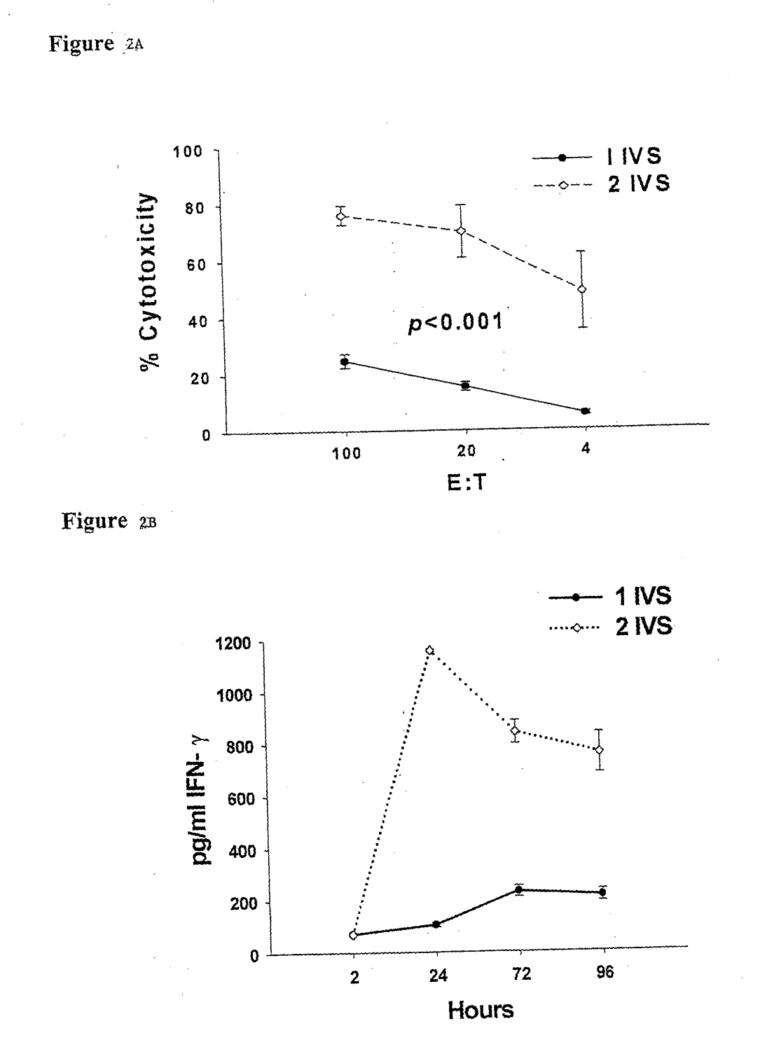

IFN-γ release is a reliable indicator of T_{H}1 responses stimulated by vaccines or vaccine candidates. Transgenic HLA A2/K^{b} mice were immunized with 100 nmol of K25V. IFN-γ release was quantitated in supernatants of splenocyte cultures after one or two in vitro stimulations. IFN-γ secretion in in vitro stimulated culture supernatants was measured by ELISA using known methods. Paired capture (anti-IFN-γ R4-6A2) and detecting (anti-IFN-γ biotinylated XMG1.2) monoclonal antibodies were obtained from Pharmingen, San Diego, Calif., USA. See Villacres et al., J. Infect. Dis. 184:256-267, 2001. Transgenic mice were vaccinated with 100 nmol of K25V fusion peptide as described in Example 3 and boosted two weeks later with an additional 100 nmol of the identical peptide. Mice (N=8) were sacrificed after two weeks, spleens removed, and either one (filled circles) or two (open diamonds) in vitro stimulations were performed followed by a chromium release assay as described in Example 4. Cytotoxicity results are presented in FIG. 2A.

Values represent subtraction of non-specific (p53₁₄₉₋₁₅₇) from specific (pp65₄₉₅₋₅₀₃) cytotoxicity of peptide-sensitized T2 cells. One in vitro stimulation resulted in modest IFN-γ release and corresponding cytotoxicity (FIG. 2B), while a second in vitro stimulation dramatically improved the IFN-γ signal and cytotoxicity. See FIGS. 2A, 2B. K25V fusion peptide therefore has favorable solubility and activity characteristics in physiologic saline with minimal DMSO.

Aliquots of culture medium (200 μl) from in vitro stimulated cultures (filled circles, one in vitro stimulation; open diamonds, two in vitro stimulations) from mice immunized as described above were withdrawn at the indicated times, and IFN-γ protein was measured from the undiluted fluid by ELISA. Recombinant IFN-γ (Pharmingen, San Diego, Calif., USA) was used to prepare a standard curve. Each sample was tested in duplicate. The detection limit of the assay was established as 70 pg/ml using IFN-γ protein standard (Pharmingen).

### Example 6

### Cytofluorimetric Analysis

A HLA-A2 CMVpp65₄₉₅₋₅₀₃ tetramer reagent was refolded and purified using aminor modification of the procedure used by the NIAID Tetramer Core Facility (www.emor.edu/WHSC/TETRAMER). HLA-A2 heavy chain and beta-2-microglobulin (β₁, M), cloned in the vector pHNl, were expressed in E. coli XA90 and refolded with the CMV pp65₄₉₅₋₅₀₃ CTL epitope. See Villacres et al., J. Infect. Dis. 184:256-267, 2001. The refolded HLA-A2/β₁M/peptide complexes were biotinylated using the enzyme BirA (Avidity Inc., Denver, Colo., USA), and then purified by FPLC chromatography using a Sephacryl S300 gel filtration column and then a MonoQ ion exchange column. The purified biotinylated HLA-A2/β₁M/peptide complexes were conjugated to either streptavidin-PE (Pharmingen, San Diego, Calif., USA) or to streptavidin-APC (Molecular Probes, Eugene, Oreg., USA). Labeling was typically performed using 0.5 μg of tetramer to stain 0.5 to 1 million cells in a 50-100 μl volume of PBS/0.5% BSA for 20 minutes. The cells were then washed and analyzed on a Becton-Dickinson FACScalibur T flow cytometer (Franklin Lakes, N.J., USA). A lymphocyte gate was set based on forward and side scatter and a minimum of 30,000 gated events captured. Quadrants were set based on negative controls. The number of tetramer-positive cells was expressed as a percentage of the total lymphocyte population.

### Example 7

### Immunogencity of Fusion Peptides with CPG ssODN

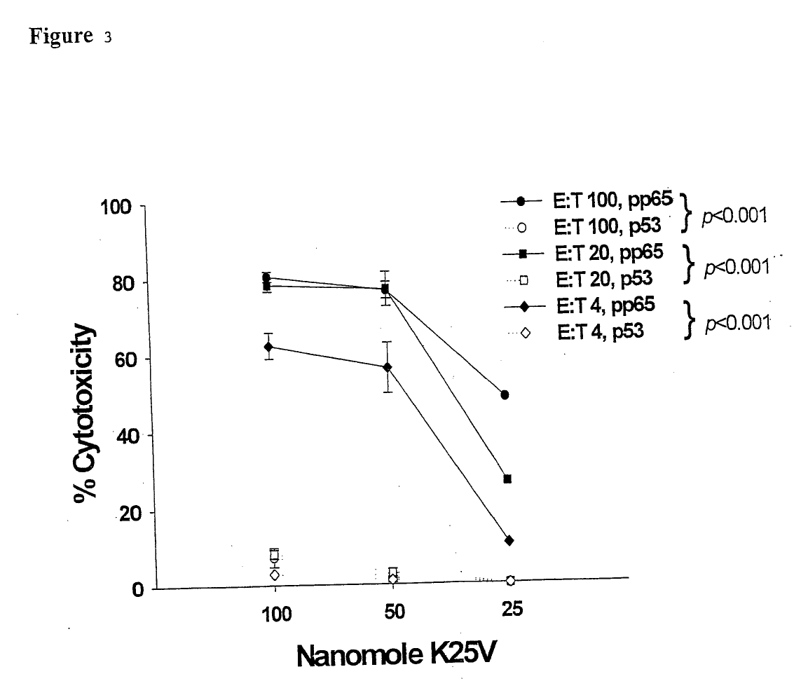

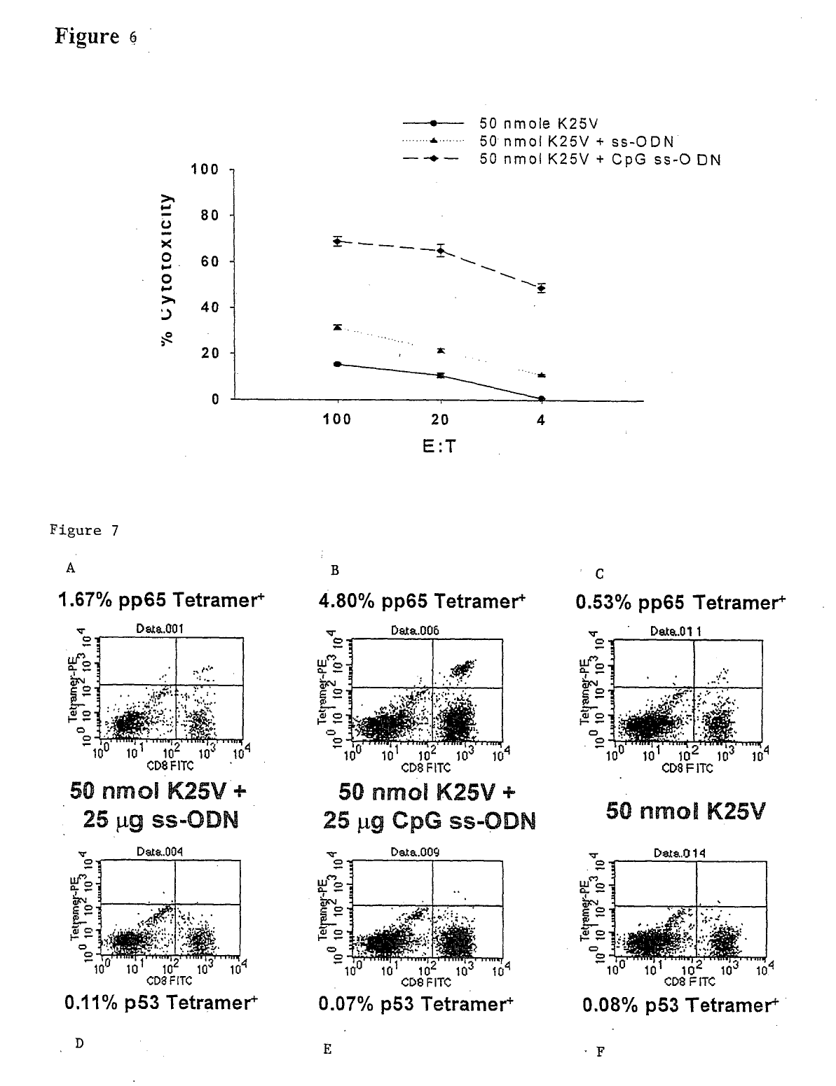

K25V (prepared as in Example 3) was mixed with CpG-containing ss-ODN referred to as #1826 (25 μg) in NSD. A dose titration of peptide was set-up, with a constant volume maintained by dilution with N-saline. One hundred microliters of peptide/25 μg ss-ODN solution was injected once subcutaneously into transgenic mice in the following groups (N=6 (100 nmol), 10 (50 nmol), 2 (25 nmol). Two weeks later, spleens were removed and one in vitro stimulation was performed as described in Example 3. A chromium release assay as described in Example 4 demonstrated cytotoxicity of the CMV-specific cells. See FIG. 3. pp65₄₉₅₋₅₀₃ cytotoxicity is represented by filled symbols, and p53₁₄₉₋₁₅₇ (a control peptide) specificity is represented by open symbols. Targets and calculation of cytotoxicity were the same as described in Example 3. Compared to mice immunized without CpG ss-ODN in which only one IVS amplification was performed (FIG. 2A), there is substantial upregulation of peptide-specific recognition in the presence of CpG ss-ODN in combination with either 50 or 100 nmol fusion peptide. See FIG. 3. The dramatic effect of ss-ODN is not observed when a non-CpG ss-ODN (#1982) is used (see FIG. 6).

### Example 8

### Tetanus T_{H} Epitopes as Part of Fusion Peptides Mediate Potent Cytotoxic Responses

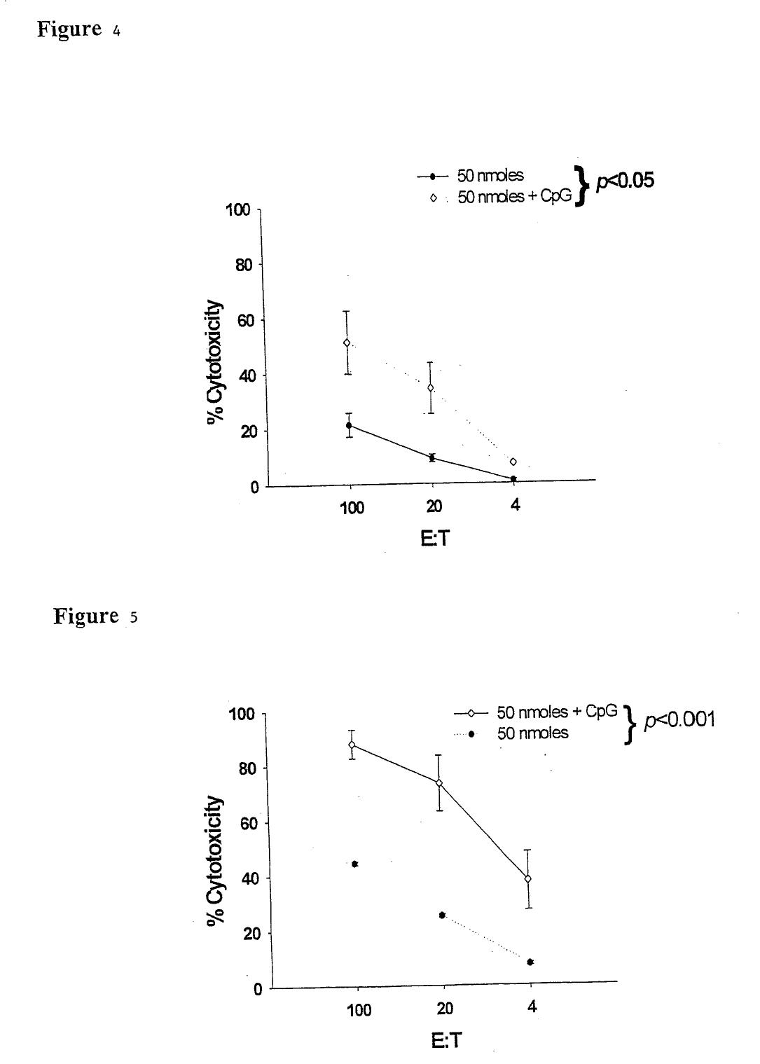

The effect of CpG ss-ODN was also investigated in combination with two other fusion peptides, both containing promiscuous T_{H} epitopes from tetanus (See Table I). The KTet₈₃₀V fusion peptide was given by subcutaneous injection with (FIG. 4, open diamonds) and without (FIG. 4, filled circles) ss-ODN, and the CTL response was evaluated as described in Example 8. KTet₈₃₀ V given to mice at either 50 (FIG. 4) or 100 (data not shown) nmol was not able to stimulate a vigorous CTL response without CpG ss-ODN adjuvant. A similar effect was observed with another tetanus T_{H} epitope called Tet₆₃₉V although the effect of CpG ss-ODN was not as dramatic (FIG. 5). In FIG. 5, the data represent results under the same conditions as FIG. 4 (N=4) except the fusion peptide is Tet₆₃₉ V (see Table I) and symbols represent 50 nmol of peptide alone (filled circles) or with 25 μg ss-ODN #1826 (open diamonds). Standard measures of hydrophobicity indicate that Tet₆₃₉ is similarly hydrophobic as PADRE, but Tet₈₃₀ is more hydrophilic. Kyte and Doolittle, J. Mol. Biol. 157:105-132, 1982; Sweet and Eisenberg, J. Mol. Biol. 171:479-488, 1983. The data shows that several T_{H} epitopes can substitute for PADRE, but the degree of hydrophobicity may be important for both CTL-stimulating activity generally and the ability of CpG ss-ODN to upregulate function.

### Example 9

### Immunization Strength Analysis with HIA A2.1 Tetramer Reagent

HLA tetramers are an independent means of assessing CTL frequency. They provide a direct, quantitative measure of the frequency of peptide-specific CTL without relying on limiting dilution or in vitro culture methods. See Villacres et al., J. Infect. Dis. 184:256-267, 2001; Altman et al., Science 274:94-96, 1996; Appay et al., J. Exp. Med. 192:63-74, 2000. The same tetramer preparation that worked specifically with human PBMC distinguishes pp65₄₉₅₋₅₀₃-specific T cells from mouse spleen, as was recently shown for a human p53 HLA A2.1 CTL epitope. See Hernandez et al., J. Immunol. 164:596-602, 2000. Here, three groups of transgenic mice were immunized subcutaneously with 50 nmol K25V and a booster of the same composition, with either control (non-CpG; FIG. 6, filled triangles), or CpG ss-ODN (FIG. 6, filled diamonds), or alone (FIG. 6, filled circles). Spleens were harvested after 14 days, and one in vitro stimulation was performed. Conditions for chromium release assay and calculation of specific cytotoxicity were as described in Example 5. Cytotoxicity data for the booster immunization are shown in FIG. 6. The results of the primary immunization are consistent with those shown in FIGS. 2A and 3. The fusion peptide was recognized and produced cytotoxicity independent of DNA adjuvant, although ss-ODN, especially CpG-containing DNA, upregulated the activity. The adjuvant effect was most apparent after a second administration of vaccine.

Phycoerythrin (PE)-HLA tetramer two-color flow cytometry, visualized with FITC-CD8 is shown in FIG. 7 for all three groups of transgenic mice. See FIGS. 7A-7C (specific HLA tetramer pp65₄₉₅₋₅₀₃) or FIGS. 7D-7F (non-specific HLA tetramer pp65₁₄₉₋₁₅₇). Percentages of cells which are in the top-right quadrant are shown for each profile. Twenty thousand events were collected for each histogram, and electronic gates were used to exclude cells that did not fall into the small lymphocyte size range. Levels of stained T cells and cytotoxicity correlated well. Insubstantial background staining with the non-specific HLA-tetramer indicates specificity of the interaction.

### Example 10

### Mucosal Administration of K25V Vaccine

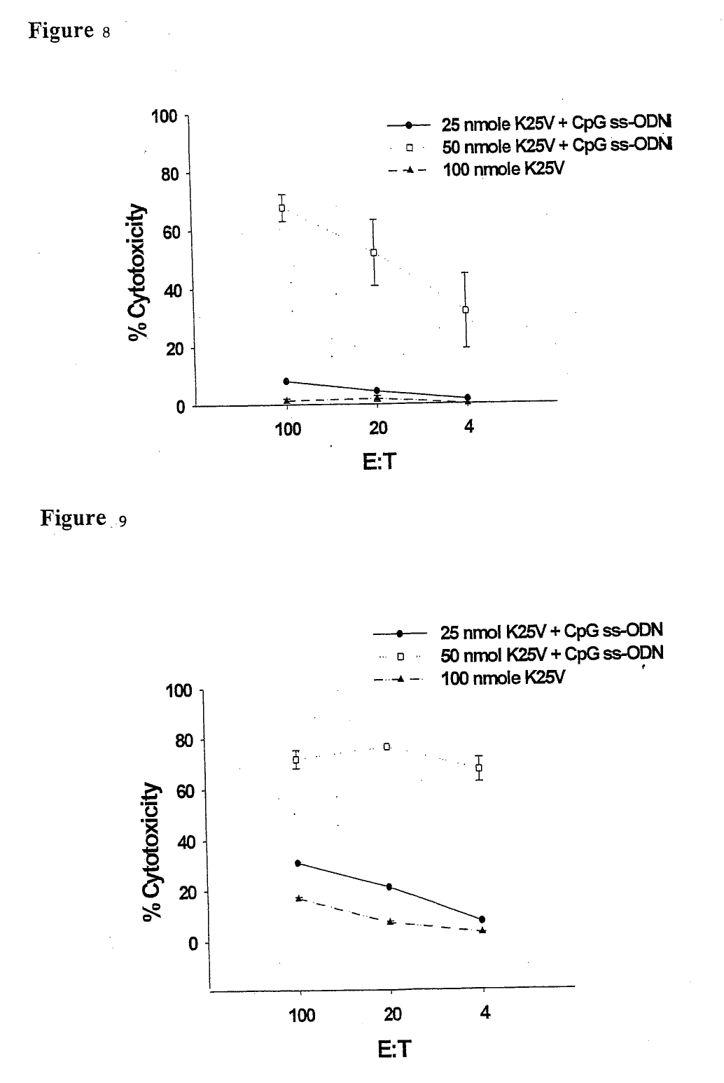

Mucosal immunization of fusion peptides was carried out, using splenic lymphocytes to evaluate whether this route of administration produced systemic immunity. Free peptides generally have not been effective immunogens when introduced by the mucosal route. CpG ss-ODN, however, can be effective adjuvants using protein immunogens when administered mucosally. See Homer et al., J. Immunol. 167:1584-1591, 2001; McCluskie et al., Vaccine 19:3759-3768, 2001. K25V was administered intranasally to transgenic HLA A2.1/K^{b} mice, either alone or mixed with CpG ss-ODN as described in Example 4 (15 μl was introduced into each nare under anesthesia, for a total of 30 μl total/mouse). FIG. 8 shows cytotoxicity of splenocytes from mice immunized once with the indicated immunogen and sacrificed two weeks later. FIG. 9 shows cytotoxicity of splenocytes immunized twice with the indicated immunogen and sacrificed three weeks later. All splenocytes were subjected to in vitro stimulation as described in Example 4 for seven days and then assayed for cytotoxicity by chromium release as described for subcutaneous immunizations (see Example 5). The 25 or 50 nmol doses with DNA were effective at stimulating CTL. One hundred nanomole doses without DNA demonstrated some activity (see FIG. 9). In contrast to the subcutaneous route (see FIG. 2A), activity of peptide delivered by the intranasal route shows striking dependence on CpG ss-ODN.

### Example 11

### Fusion Peptide Directs CTL Recognition of Endogenously Processed pp65 Protein

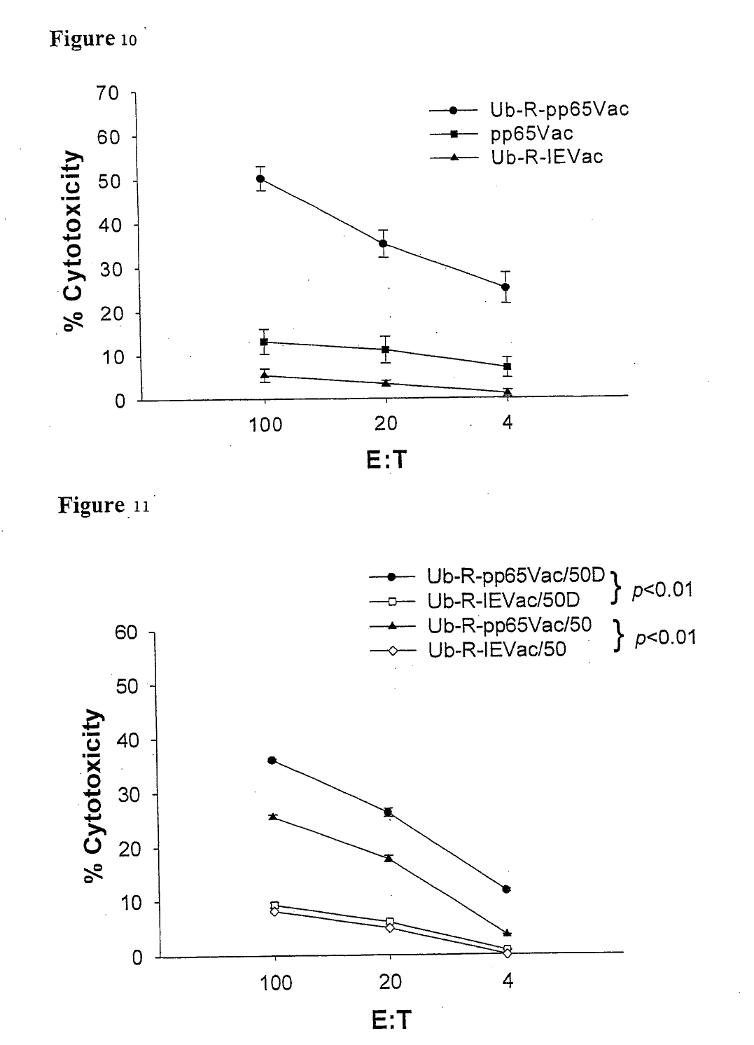

An in vitro model was designed to evaluate recognition of virally expressed pp65. Human Jurkat T cells stably expressing HLA A2.1 (JA2.1) were infected with pp65 expressed in vaccinia virus. In contrast to peptide-loaded T2 targets, virally infected targets support TAP-dependent protein processing for successful recognition. Wei et al., Nature 356:443-446, 1992. A bulk spleen cell culture derived from the K25V immunization shown in FIG. 1 after repeated (5×) in vitro stimulation with K25V (FIG. 1, 100 nmole) was used to evaluate the efficiency of recognition of pp65Vac. CRA targets (JA2.1 T cells) were either infected with vaccinia virus expressing Ub-R-pp65Vac (filled circles), pp65Vac (filled squares) or Ub-R-IEVac (filled triangles) for 16 hours at an MOI of 3 (FIG. 10) or pulsed with peptides (data not shown). Little recognition of pp65Vac-infected targets was observed using the bulk line (FIG. 10), whereas pp65₄₉₅₋₅₀₃ loaded JA2.1 (data not shown) was recognized comparably to that of T2 cells (FIG. 1, 100 nmole). Human pp65-specific CTL recognize pp65Vac-infected targets very efficiently. Diamond et al., Blood 90:1751-1767, 1997. Non-specific lysis is shown (Ub-R-IEVac, filled triangles) for VV-infected targets, and was <5% for peptide-loaded T2 cells (data not shown). Error bars represent averages of 4 separate experiments carried out on different days. A double knock-out transgenic mouse devoid of H-2 Ia expression has facilitated greater recognition of HLA A2.1/restricted antigens. Ureta-Vidal et al., J. Immunol. 163:2555-2560, 1999. A destabilized form of the pp65 protein was engineered according to the N-end Rule model described by Varshavsky and collaborators. See Tobery and Siliciano, J. Exp. Med. 185:909-920, 1997; Varshavsky, Proc. Natl. Acad. Sci. U.S.A. 93:12142-12139, 1996; Rock and Goldberg, 17:739-779, 1999. When JA2.1 cells infected with ubiquitinated pp65 (Ub-R-pp65Vac) is the target, a significant cytolytic response is detectable compared to unmodified pp65 (FIG. 10). The specificity of the response against pp65 has been confirmed; a non-specific ubiquitinated protein from CMV was recognized only minimally (FIG. 10).

To evaluate whether CTL stimulated by fusion peptide can recognize full length pp65, 50 nmol Tet₆₃₉V, alone or with 25 μg CpG ss-ODN, was administered subcutaneously. A chromium release assay was carried out on splenocytes after a single primary immunization. Targets were either JA2.1 T cells infected with Ub-R-pp65Vac (filled circles or triangles), or Ub-R-IEVac (filled squares and diamonds) as described in Example 3. As expected, peptide-specific responses were easily measured after one immunization for both preparations (FIG. 5), but recognition of endogenously processed pp65 was also evident, and more prominent with the preparation containing DNA (FIG. 11). This confirms that fusion peptides delivered subcutaneously stimulate CTL that recognize processed full length pp65. A similar result obtained when 50 nmol K25V and 25 or even 10 μg CpG ss-ODN were administered subcutaneously or intranasally (data not shown). The addition of CpG ss-ODN had a major effect on recognition of full length pp65, as shown in FIG. 11. This effect was also found after intranasal administration, since 100 nmol K25V gave a good peptide-specific response, but co-administered CpG ss-ODN was required to detect recognition of full-length pp65Vac (data not shown).

### REFERENCES

1. Bernhard, et al., “Cytotoxic T lymphocytes from HLA-A2 transgenic mice specific for HLA-A2 expressed on human cells.” J. Exp. Med. 168: 1157-1162, 1988.

2. Rosloniec, et al., “An HLA-DR1 trans gene confers susceptibility to collagen-induced arthritis elicited with human type II collagen.” J. Exp. Med. 185:1113-1122, 1997.

3. Wills, et al., “The human CTL response to cytomegalovirus is dominated by structural protein pp65: Frequency, specificity, and T Cell Receptor usage of pp65-Specific CTL.” J. Virol. 70:7569-7579, 1996.

4. Longmate et al., “Population coverage by HLA class-I restricted cytotoxic T-lymphocyte epitopes.” Immunogenetics 52:165-173, 2001.

5. Nichols et al., “High Risk of Death Due to Bacterial and Fungal Infection among Cytomegalovirus (CMV)-Seronegative Recipients of Stem Cell Transplants from Seropositive Donors: Evidence for Indirect Effects of Primary CMV Infection.” J. Infect. Dis. 185:273-282, 2002.

6. Limaye et al., “High Incidence of Ganciclovir-Resistant Cytomegalovirus Infection among Lung Transplant Recipients Receiving Preemptive Therapy.” J. Infect. Dis. 185:20-27, 2002.

7. Stratton et al., “A tool for Decisionmaking.” Bethesda, National Academy Press, 2001.

8. Nichols et al., “Rising pp65 antigenemia during preemptive anticytomegalovirus therapy after allogeneic hematopoietic stem cell transplantation: risk factors, correlation with DNA load, and outcomes.” Blood 97:867-874, 2001.

9. Zaia et al., “Status of cytomegalovirus prevention and treatment in 2000.” Hematology (Am. Soc. Hematol. Educ. Program.):339-355, 2000.

10. Krause et al., “Screening for CMV-specific T cell proliferation to identify patients at risk of developing late onset CMV disease.” Bone Marrow Transplant 19:1111-1116. 1997.

11. Grefte et al., “The lower matrix protein pp65 is the principal viral antigen present in peripheral blood leukocytes during an active cytomegalovirus infection.” J. Gen. Virol. 73:2923-2932, 1992.

12. Riddell et al., “Class I MHC-restricted cytotoxic T lymphocyte recognition of cells infected with human cytomegalovirus does not require endogenous viral gene expression.” J. Immunol. 146:2795-2804, 1991.

13. Diamond et al., Development of a candidate HLA A*0201 restricted peptide-based vaccine against human cytomegalovirus infection.” Blood 90:1751-1767, 1997.

14. BenMohamed et al., “Intranasal Administration Of A Synthetic Lipopeptide Without Adjuvant Induces Systemic Immune Responses.” Immunology 106:113-121, 2002.

15. BenMohamed et al., “Induction of CTL Response by a Minimal Epitope Vaccine in HLA A*020 1/DR1 Transgenic Mice: Dependence on HLA Class II Restricted T_{H} Response.” Hum. Immunol. 61:764-779, 2002.

16. Hioe et al., “Comparison of adjuvant formulations for cytotoxic T cell induction using synthetic peptides.” Vaccine 14:412-418, 1996.

17. Mora et al., “Controlled lipidation and encapsulation of peptides as a useful approach to mucosal immunizations.” J. Immunol. 161:3616-3623, 1998.

18. Partidos et al., “CTL responses induced by a single immunization with peptide encapsulated in biodegradable microparticles.” J. Immunol. Meth. 206:143-151, 1997.

19. Hart et al., “Priming of anti-human immunodeficiency virus (HIV) CD8+cytotoxic T cells in vivo by carrier-free HIV synthetic peptides.” Proc. Natl. Acad. Sci. U.S.A 88:9448-9452, 1991.

20. Shirai et al., “Helper-cytotoxic T lymphocyte (CTL) determinant linkage required for priming of anti-HIV CD8+ CTL in vivo with peptide vaccine constructs.” J. Immunol. 152:549-556, 1994.

21. Livingston et al., “The Hepatitis B virus-specific CTL responses induced in humans by lipopeptide vaccination are comparable to those elicited by acute viral infection.” J. Immunol. 159:1383-1392, 1997.

22. Martinon et al., “Immunization of mice with lipopeptides bypasses the prerequisite for adjuvant. Immune response of BALB/c mice to human immunodeficiency virus envelope glycoprotein.” J. Immunol. 149:3416-3422, 1992.

23. Heathcote et al., “A Pilot Study of the CY-1899 T-Cell Vaccine in Subjects Chronically Infected With Hepatitis B Virus.” Hepatology 30:531-536, 1999.

24. Gahery-Segard et al., “Multiepitopic B- and T-cell responses in humans by a HIV Type 1 lipopeptide vaccine.” J. Virol. 74:1694-1703, 2000.

25. Seth et al., “Evaluation of a lipopeptide immunogen as a therapeutic in HIV type 1-seropositive individuals.” AIDS Res. Hum. Retroviruses 16:337-343, 2000.

26. Banchereau et al., “Immune and clinical responses in patients with metastatic melanoma to CD34(+) progenitor-derived dendritic cell vaccine.” Cancer Res. 61:6451-6458, 2001.

27. Ludewig et al., “Dendritic cells efficiently induce protective antiviral immunity.” J. Virol. 72:3812-3818, 1998.

28. Freytag and Clements, “Bacterial toxins as mucosal adjuvants.” Curr. Top. Microbiol. Immunol. 236:215-236, 1999.

29. Newman et al., “Induction of cross-reactive cytotoxic T-lymphocyte responses specific for HIV-1 gp120 using saponin adjuvant (QS-21) supplemented subunit vaccine formulations.” Vaccine 15:1001-1007, 1997.

30. Wiedemann et al., “Histopathological studies on the local reactions induced by complete Freund's adjuvant (CPA), bacterial lipopolysaccharide (LPS), and synthetic lipopeptide (P3C) conjugates.” J. Pathol. 164:265-271, 1991.

31. Belyakov et al., “Mucosal AIDS vaccine reduces disease and viral load in gut reservoir and blood after mucosal infection of macaques.” Nat. Med. 7:1320-1326, 2001.

32. Krieg et al., “Sequence motifs in adenoviral DNA block immune activation by stimulatory CpG motifs.” Proc. Natl. Acad. Sci. U.S.A. 95:12631-12636, 1998.

33. Boyer et al., “Vaccination of seronegative volunteers with a human immunodeficiency virus type 1 env/rev DNA vaccine induces antigen-specific proliferation and lymphocyte production of beta-chemokines.” J. Infect. Dis. 181:476-483, 2000.

34. Berger et al., “Nonmyeloablative immunosuppressive regimen prolongs In vivo persistence of gene-modified autologous T cells in a nonhuman primate model.” J. Virol. 75:799-808, 2001.

35. Davis et al., “CpG DNA is a potent enhancer of specific immunity in mice immunized with recombinant hepatitis B surface antigen.” J. Immunol. 160:870-876, 1998.

36. Homer et al., “Immunostimulatory DNA-based vaccines elicit multifaceted immune responses against HIV at systemic and mucosal sites.” J. Immunol. 167:1584-1591, 2001.

37. Brazolot Millan et al., “CpG DNA can induce strong Th1 humoral and cell-mediated immune responses against hepatitis B surface antigen in young mice.” Proc. Natl. Acad. Sci. U.S.A. 95:15553-15558, 1998.

38. Alexander et al., “Development of high potency universal DR-restricted helper epitopes by modification of high affinity DR-blocking peptides.” Immunity 1:751-761, 1994.

39. La Rosa et al., “Enhanced immune activity of cytotoxic T-lymphocyte epitope analogs derived from positional scanning synthetic combinatorial libraries.” Blood 97:1776-1786, 2001.

40. Lipford et al., “CpG-containing synthetic oligonucleotides promote B and cytotoxic T cell responses to protein antigen: a new class of vaccine adjuvants.” Eur. J. Immunol. 27:2340-2344, 1997.

41. Krieg et al., “CpG motifs in bacterial DNA trigger direct B-cell activation.” Nature 374:546-549, 1995.

42. Tobery and Siliciano, “Targeting of HIV-1 antigens for rapid intracellular degradation enhances cytotoxic T lymphocyte (CTL) recognition and the induction of de novo CTL responses in vivo after immunization.” J. Exp. Med. 185:909-920, 1997.

43. Yao et al., “Site-directed mutation in a conserved kinase domain of human cytomegalovirus-pp65 with preservation of cytotoxic T lymphocyte targeting.” Vaccine 19:1628-1635, 2001.

44. Chakrabarti et al., “Vaccinia virus expression vector: coexpression of beta-galactosidase provides visual screening of recombinant virus plaques.” Mol. Cell. Biol. 5:3403-3409, 1985.

45. Vitiello et al., “Comparison of cytotoxic T lymphocyte responses induced by peptide or DNA immunization: implications on immunogenicity and immunodominance.” Eur. J. Immunol. 27:671-678, 1997.

46. Wei and Cresswell, “HLA-A2 molecules in an antigen-processing mutant cell contain signal sequence-derived peptides.” Nature 356:443-446, 1992.

47. Villacres et al., “Human immunodeficiency virus-infected patients receiving highly active antiretroviral therapy maintain activated CD8+T cell subsets as a strong adaptive immune response to cytomegalovirus.” J. Infect. Dis. 184:256-267, 2001.

48. Sauzet et al., “Long-lasting anti-viral cytotoxic T lymphocytes induced in vivo with chimeric-multirestricted lipopeptides.” Vaccine 13:1339-1345, 1995.

49. Schild et al., “Efficiency of pep tides and lipopeptides for in vivo priming of virus-specific cytotoxic T cells.” Eur. J. Immunol. 21:2649-2654, 1991.

50. Kyte and Doolittle, “A simple method for displaying the hydropathic character of a protein.” J. Mol. Biol. 157:105-132, 1982.

51. Sweet and Eisenberg, “Correlation of sequence hydrophobicities measures similarity in three-dimensional protein structure.” J. Mol. Biol. 171:479-488, 1983.

52. Tsunoda et al., “Lipopeptide particles as the immunologically active component of CTL inducing vaccines.” Vaccine 17:675-685, 1999.

53. Klinman et al., “CpG DNA augments the immunogenicity of plasmid DNA vaccines.” Curr. Top. Microbiol. Immunol. 247:131-142, 2000.

54. Tighe et al., “Conjugation of protein to immunostimulatory DNA results in a rapid, long-lasting and potent induction of cell-mediated and humoral immunity.” Eur. J. Immunol. 30:1939-1947, 2000.

55. Chu et al., “CpG oligodeoxynucleotides act as adjuvants that switch on T helper 1 (Th1) immunity.” J. Exp. Med. 186:1623-1631, 1997.

56. Altman et al., “Phenotypic analysis of antigen-specific T lymphocytes.” Science 274:94-96, 1996.

57. Appay et al., “HIV-specific CD8(+) T cells produce antiviralcytokines but are impaired in cytolytic function.” J. Exp. Med. 192:63-75, 2000.

58. Hernandez et al., “The use of HLAA2.1/p53 peptide tetramers to visualize the impact of self tolerance on the TCR repertoire.” J. Immunol. 164:596-602, 2000.

59. Morris et al., “Effectiveness of intranasal immunization with HIV gp160 and an HIV-1 env CTL epitope peptide (E7) in combination with the mucosal adjuvant L T(RI92G).” Vaccine 18:1944-1951, 2000.

60. McCluskie et al., “Mucosal immunization of mice using CpA DNA and/or mutants of the heat-labile enterotoxin of Escherichia coli as adjuvants.” Vaccine 19:3759-3768, 2001.

61. Ureta-Vidal et al., “Phenotypical and functional characterization of the CD8+T cell repertoire of HLA-A2.1 transgenic, H-2 KbnullDbnull double knockout mice.” J. Immunol. 163:2555-2560, 1999.

62. Varshavsky, “The N-end rule: functions, mysteries, uses.” Proc. Natl. Acad. Sci. U.S.A. 93:12142-12149, 1996.

63. Rock and Goldberg, “Degradation of cell proteins and the generation of MHC class 1-presented peptides.” Annu. Rev. Immunol. 17:739-779, 1999.

64. Alexander et al., “Derivation of HLA-A11/K^{b} transgenic mice: functional CTL repertoire and recognition of human All-restricted CTL epitopes.” J. Immunol. 159:4753-4761, 1997.

65. Gratama et al., “Tetramer-based quantification of cytomegalovirus (CMV)-specific CD8+ T lymphocytes in T-cell-depleted stem cell grafts and after transplantation may identify patients at risk for progressive CMV infection.” Blood 98:1358-1364, 2001.

66. Cwynarski et al., “Direct visualization of cytomegalovirus-specific T-cell reconstitution after allogeneic stem cell transplantation.” Blood 97:1232-1240, 2001.

67. Walter et al., “Reconstitution of cellular immunity against cytomegalovirus in recipients of allogeneic bone marrow by transfer of T-cell clones from the donor.” N. Engl. J. Med. 333:1038-1044, 1995.

68. Einsele et al., “Infusion of cytomegalovirus (CMV)-specific T cells for the treatment of CMV infection not responding to antiviral chemotherapy.” Blood 99:3916-3922, 2002.

69. Pepperl et al., “Dense bodies of human cytomegalovirus induce both humoral and cellular immune responses in the absence of viral gene expression.” J. Virol. 74:6132-6146, 2000.

70. Li et al., “Recovery of HLA-restricted cytomegalovirus (CMV)-specific T-cell responses after allogeneic bone marrow transplant; correlation with CMV disease and effect of ganciclovir prophylaxis.” Blood 83:1971-1979, 1994.

71. Reece et al., “Mapping the major human T helper epitopes of tetanus toxin. The emerging picture.” J. Immunol. 151:6175-6184, 1993.

1. A cytomegalovirus vaccine which comprises a fusion peptide composed of a T helper epitope fused to a CMV CTL epitope peptide.

2. A cytomegalovirus vaccine of claim 1 wherein said T helper epitope is PADRE.

3. A cytomegalovirus vaccine of claim 1 wherein said T helper epitope is a tetanus epitope.

4. A cytomegalovirus vaccine of claim 3 wherein said tetanus epitope is selected from the group consisting of tetanus heavy chain (590-603), tetanus heavy chain (615-629), tetanus heavy chain (639-652), tetanus heavy chain (830-843), and tetanus heavy chain (947-967).

5. A cytomegalovirus vaccine of claim 1 wherein said CMV pp65 CTL epitope peptide is selected from the group consisting of pp65₁₃₋₂₄, pp65₁₈₆₋₁₉₆, pp65₁₈₈₋₁₉₅, pp65₂₆₅₋₂₇₅, Pp65₃₆₃₋₃₇₃, pp65₃₆₉₋₃₇₉, pp65₃₆₇₋₃₇₉, pp65₄₉₅₋₅₀₃ and pp65_{417-426.}

6. A cytomegalovirus vaccine of claim 5 wherein said CMV pp65 CTL epitope is pp65_{495-503.}

7. A cytomegalovirus vaccine of claim 1 which further comprises a DNA adjuvant.

8. A cytomegalovirus vaccine of claim 6 wherein said DNA adjuvant is selected from the group consisting of SEQ ID NO:8, SEQ ID NO:9 and SEQ ID NO:10.

9. A cytomegalovirus vaccine of claim 1 which further comprises a pharmaceutically acceptable carrier.

10. A fusion peptide comprising a T helper epitope fused to a CMV CTL epitope peptide.

11. A fusion peptide of claim 10 wherein said T helper epitope is PADRE.

12. A fusion peptide of claim 10 wherein said T helper epitope is a tetanus epitope.

13. A fusion peptide of claim 10 wherein said tetanus epitope is selected from the group consisting of tetanus heavy chain (590-603), tetanus heavy chain (615-629), tetanus heavy chain (639-652), tetanus heavy chain (830-843), and tetanus heavy chain (947-967).

14. A method of modifying the immune response of a mammal to CMV comprising administering an effective amount of a vaccine of claim 1.
